(19) United States
(12) Patent Application Publication
(10) Pub. No.: US 2026/0283737 A1
(43) Pub. Date: Sep. 24, 2026

(54) COMPLETE MEDICAL SYSTEM AND METHOD FOR IMAGE SUPPORT

(71) Applicant: Siemens Healthineers AG, Forchheim (DE)

(72) Inventors: Alois Regensburger, Poxdorf (DE); Marcus Pfister, Bubenreuth (DE)

(21) Appl. No.: 19/559,276

(22) Filed: Mar. 6, 2026

(30) Foreign Application Priority Data
Mar. 20, 2025 (DE) 10 2025 110 903.6

(51) Int. Cl. A61B 90/00; A61B 34/20; A61B 34/30; A61B 34/32

(52) U.S. Cl. CPC A61B 90/37; A61B 34/20; A61B 34/32; A61B 2034/2051; A61B 2034/2065; A61B 2034/301; A61B 2090/3735; A61B 2090/3764; A61B 2090/378

(57) ABSTRACT

A method for image support for navigation of an instrument having an imaging apparatus in a hollow organ section surrounded by at least one ring-shaped muscle or tissue structure includes: capturing or providing at least one intraluminal image of the hollow organ section, which was captured by the imaging apparatus; detecting the muscle or tissue fibers surrounding the hollow organ section and/or aligning the muscle or tissue fibers of at least one muscle or tissue structure of the hollow organ section based on at least one image; ascertaining the current position and/or orientation of the instrument and/or the tip of the instrument; and determining and providing a model and/or visualization of the muscle or tissue fibers of the hollow organ section relative to the current position and/or orientation of the instrument.

The present patent document claims the benefit of German Patent Application No. 10 2025 110 903.6, filed Mar. 20, 2025, which is hereby incorporated by reference in its entirety.

### TECHNICAL FIELD

The disclosure relates to a complete medical system designed to execute a method for image support for navigation of an instrument including an imaging apparatus in a hollow organ section surrounded by at least one ring-shaped muscle or tissue structure, in particular a sphincter muscle. The disclosure also relates to a method for image support for navigation of an instrument including an imaging apparatus in a hollow organ section surrounded by at least one ring-shaped muscle or tissue structure, in particular a sphincter muscle.

### BACKGROUND

Endoscopic retrograde cholangiopancreatography (ERCP) canulation, (i.e., the insertion of an instrument such as a cannula or a catheter in a human bile duct for an examination), poses a number of challenges for the treating physician. It is challenging, for example, that the injection of a contrast agent that makes visualization of the bile duct significantly easier may be avoided as the contrast agent may cause complications such as pancreatitis. Canulation is performed “blind” because without contrast agent only the instrument, and not the bile ducts, may be discerned in the X-ray image. Especially for inexperienced doctors or in cases of difficult patient anatomy and clinical pictures such as obstructions and swelling, this procedure may be protracted and stressful for the patient. In the event of difficult canulation with many failed attempts, side effects such as pancreatitis and even bleeding in the bile duct occur in many cases.

Anatomically, the sections of the bile duct relevant for canulation are sphincter muscles (sphincters), similar to those found, for example, in the esophagus or intestinal tract. In heathy anatomy, these sphincters control the transport of bile (bile duct fluid) from the common bile duct and the pancreatic duct and prevent unwanted reflux of material from the small intestine. The sphincter muscles are essentially arranged in a ring around the bile duct section. However, due to the groove-shaped structure and the partial contraction of the muscle fibers, the corresponding instrument (or other canulation instrument) may partially catch in the sphincters and, instead of continuing to slide along the bile duct, irritate or injure the vessel wall.

The following methods for solving the problem are known from the prior art. Preventive treatment of the consequences of tissue irritation may be performed by suboptimal canulation (e.g., preventive stent in the pancreas). Muscle relaxants may be administered, but they are associated with side effects and are not always successful. Imaging (e.g., diagnostic imaging) with OCT may be used in the bile duct. If canulation fails, invasive methods (e.g., incision of the bile ducts, percutaneous puncture) are available, each of which represents an additional impairment and an additional risk of complications for the patient. Furthermore, referral to a specialized treatment center is possible, but this results in additional costs and additional stress for the patient.

### SUMMARY AND DESCRIPTION

The object of the present disclosure is to provide a complete medical system for executing a method which facilitates the movement of instruments, (e.g., canulation in a hollow organ with a sphincter muscle such as a bile duct), and reduces the risk of complications. Furthermore, the object of the disclosure is to provide a suitable method.

The scope of the present disclosure is defined solely by the appended claims and is not affected to any degree by the statements within this summary. The present embodiments may obviate one or more of the drawbacks or limitations in the related art.

The object is achieved by a complete medical system designed to execute a method for image support involving the movement of an instrument including an imaging apparatus in a hollow organ section surrounded by at least one ring-shaped muscle or tissue structure, in particular a sphincter muscle, and by a method for image support involving the movement of an instrument including an imaging apparatus in a hollow organ section surrounded by at least one ring-shaped muscle or tissue structure, in particular a sphincter muscle.

The disclosure includes a method for image support for navigation of an instrument including an imaging apparatus in a hollow organ section surrounded by at least one ring-shaped muscle or tissue structure, in particular a sphincter muscle. The method includes: capturing or providing at least one intraluminal image of the hollow organ section which was captured by the imaging apparatus; detecting the muscle or tissue fibers surrounding the hollow organ section, in particular sphincter muscle fibers, and/or an alignment of the muscle or tissue fibers, in particular the sphincter muscle fibers, of at least one muscle or tissue structure, in particular the sphincter muscle, of the hollow organ section on the basis of at least one image; ascertaining the current position and/or orientation of the instrument and/or the tip of the instrument; and determining and providing a model and/or visualization of the muscle or tissue fibers, in particular sphincter muscle fibers, of the hollow organ section relative to the current position and/or orientation of the instrument.

The method is particularly suitable for sphincter muscles and, by determining the sphincter muscle fibers and the relative position/orientation of the instrument to the sphincter muscle fibers and visualizing this relative position/orientation, enables a gentle approach to the planned forward movement, for example, canulation, in the area of bile ducts, for example. It becomes easier to determine optimal positions for the instrument. As a result, failed attempts when navigating in hollow organs such as bile ducts are avoided, thus minimizing health risks for the patient. This makes it easier for the treating physician to find the correct path and perform the appropriate treatment. The duration of treatment may also be significantly reduced. Consequential damage to health is avoided and the use of preventive medication may be reduced. In addition to sphincter muscles and sphincter muscle fibers, the method may also be used for a series of other ring-shaped or directional muscle or tissue structures surrounding a hollow organ, for example, the muscles responsible for intestinal peristalsis around a section of the intestine, the muscles along or around the esophagus, a mucosal structure surrounding the (paranasal) sinuses with directional tissue fibers, the muscles surrounding the veins, the muscles which move/control the venous valves, and the muscles which trigger a vasospasm.

Detection of sphincter muscle fibers and/or alignment of sphincter muscle fibers of at least one sphincter muscle of the hollow organ section on the basis of at least one image may take place, for example, as part of segmentation of the image. Sphincter muscles or sphincters are arranged, for example, in the human bile duct, in the stomach or in the intestine and have a ring-shaped, groove-like structure which may be recognized by image recognition or segmentation software.

According to one embodiment, the determination and provision of a model and/or visualization of the muscle or tissue fibers, in particular the sphincter muscle fibers, of the hollow organ section relative to the current position and/or orientation of the instrument includes the determination and provision of a center line of the hollow organ section. Ring-shaped muscle and tissue structures, in particular sphincter muscle fibers, may have a center point. In this manner, a so-called center line is produced for a hollow organ section, which may be formed from a sequence of center points of, for example, circular sphincter muscle fibers, and may be determined, for example, by an algorithm or image processing software.

According to a further embodiment, the additional act of determining path planning information for forward movement of the instrument along the center line of the hollow organ section, in particular for a position and/or orientation of the instrument to be assumed by the instrument, takes place. The path planning information includes, for example, a planned path of the instrument along the center line of the hollow organ section with the instrument accordingly aligned (along its longitudinal axis) as parallel as possible to it. In this manner, even hollow organ sections with very small cross-sections may be targeted and traversed with great precision. Known path planning software may be used here to determine the path planning information.

According to a further embodiment, instructions for manual or semi-automatic alignment of the instrument are determined and provided, e.g., to a robotic control unit of an intervention robot. In this manner, a surgeon may also target hollow organ sections with very small cross-sections easily and with great precision without damaging parts of the organ. The instructions may be given visually or acoustically, for example. In this manner, a surgeon may proceed according to the instructions, as necessary.

Provision may take place in various forms, for example, as output and display on a display unit (monitor). However, provision may also include transfer to another device, for example, a control unit of a medical intervention robot, or further processing in a calculation unit. In the case of closed-loop robotic control, for example, a movement command or alignment command for a (catheter/endoscopy) robot may be calculated.

According to a further embodiment, correction information for correcting a current path of the instrument is determined and output. Thus, information about a deviation of the instrument from the planned path may be output, for example, whether a deviation exists and what or how large it is, or other known path planning information, for example, about the length or direction of the planned path, may be output. The output may be visual or acoustic, for example.

According to a further embodiment, the method includes triggering control signals for automatic alignment, movement, or action of the instrument by a medical intervention robot in an orientation to be assumed by the instrument. Such intervention robots may be, for example, endoscopy robots, catheter robots or robotic canulation facilities.

In an advantageous manner, a plurality of intraluminal images, in particular from different projection directions, are captured and used for particularly clear visualization, for example, on a monitor or touchpad. If necessary, these images may also be reconstructed to form a 3D volume image in order to obtain a particularly good overview of the entire hollow organ section and a better representation of the ring-shaped sphincter muscle fibers. A particularly good 3D impression is obtained by using a plurality of images from significantly different projection directions.

According to a further embodiment, prior to the method the intraluminal imaging apparatus is registered with an X-ray device, for example, a C-arm X-ray device designed for fluoroscopy imaging, and a display is superimposed or performed together with an X-ray image. Registration may also take place using an X-ray device, which may capture 3D volume images, for example, a CT or CBCT. The volume images may be captured prior to the method (pre-operative images) and in addition to path planning or superimposition with the intraluminal images. In this manner, even better visualization is possible. Registration may also be performed optionally using information from an additional tracking system for the intraluminal imaging apparatus, such as electromagnetic tracking or shape sensing.

Advantageously, for particularly effective, tried and tested and low-radiation imaging, the intraluminal imaging apparatus is formed by an IVUS or OCT apparatus. IVUS imaging is a type of intraluminal ultrasound. Optical coherence tomography (OCT) is an imaging method in which 2D or 3D images of, for example, human tissue in micrometer resolution, are obtained by broadband light and a beam splitter.

The instrument may be formed, for example, by a canulation instrument, a guide wire or a catheter.

Advantageously, further path planning information for the instrument is determined.

The positioning of the instrument may be performed by known tracking systems, for example, based on electromagnetic tracking, optical tracking, RFID, radio, or even using an automatic navigation robot via its encoder.

Furthermore, the disclosure includes a complete medical system for executing the described method. The system includes a canulation instrument for navigation in a hollow organ of a patient, including an intraluminal imaging device, an image processing unit for detecting ring-shaped or directional muscle or tissue fibers surrounding the hollow organ section, in particular sphincter muscle fibers, and/or alignment of the muscle or tissue fibers, in particular the sphincter muscle fibers, of at least one muscle or tissue structure, in particular the sphincter muscle of the hollow organ section on the basis of at least one image. The system further includes a positioning unit to determine the current position and/or orientation of the instrument and/or the tip of the instrument. The system further includes a calculation unit to determine a model and/or visualization of the muscle or tissue structure, in particular sphincter muscle fibers, of the hollow organ section relative to the current position and/or orientation of the instrument. The system further includes a provision unit to provide the model or visualization. In certain examples, the complete medical system has a display unit for displaying the model or visualization. Advantageously, the complete medical system may include a medical intervention robot for automatic control of the instrument. The complete medical system may additionally include an X-ray device for taking X-ray images. A computer program product including an algorithm for automatically executing the described method is also included.

The disclosure and other advantageous embodiments are explained in more detail hereinafter with reference to diagrammatic views of embodiments shown in the drawings, without limiting the disclosure to these embodiments.

### BRIEF DESCRIPTION OF THE DRAWINGS

FIGS. 1 and 2 depict hollow organs with sphincter muscles according to the prior art.

FIG. 3 depicts an example of a sequence of acts of the method for image support involving the movement of an instrument through a hollow organ section.

FIG. 4 depicts a view of an example of an intraluminal OCT image in a sphincter muscle.

FIGS. 5 to 12 depict examples of various visualizations of sphincter muscle fibers of a hollow organ relative to the respective instrument.

FIG. 13 depicts an example of a further sequence of acts of the method for image support involving the movement of an instrument through a hollow organ section.

FIG. 14 depicts a view of an example of a complete medical system for executing the method.

### DETAILED DESCRIPTION

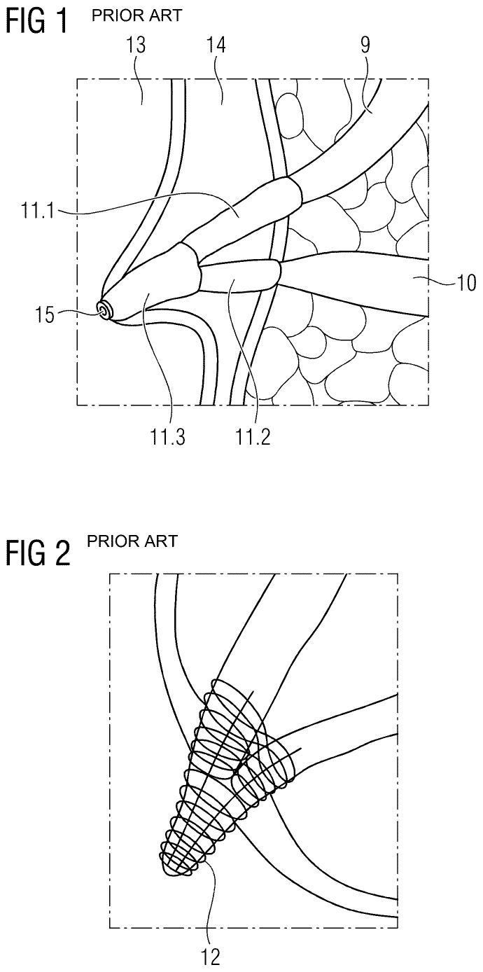

FIG. 1 shows an example of hollow organs with sphincter muscles 11 according to the prior art, the bile duct 9 and the pancreatic duct 10 together with the associated sphincter muscles 11, the choledochal sphincter 11.1, the pancreatic sphincter 11.2 and the papillary sphincter 11.3.

FIG. 2 shows the same arrangement with a focus on the sphincter muscles 11 and the sphincter muscle fibers 13. The two figures show how difficult it is to navigate an instrument correctly during an ERCP, for example, from the duodenum 13 into the very small opening 15 or inside the sections of the bile duct 9 or the pancreatic duct 10 surrounded by the sphincter muscles, above all without the use of contrast agent and without injuring the patient. The image support method offers the operator the opportunity to visualize the current position and orientation of the instrument, for example, canulation instrument or catheter, relative to the sphincter muscle fibers and thus to the ideal path, consequently simplifying planned navigation.

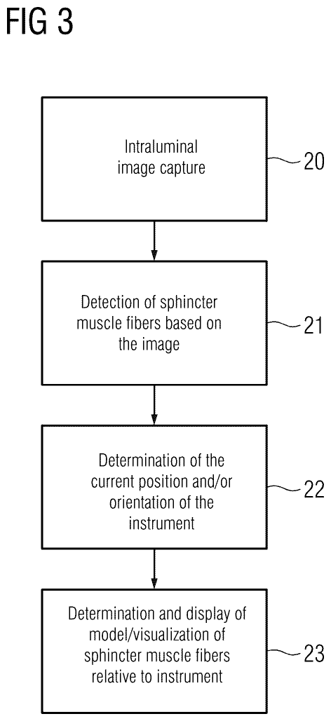

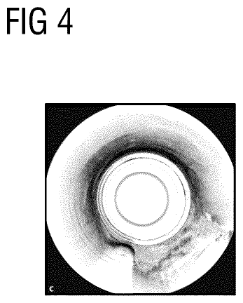

FIG. 3 shows the acts of the method. In act 20, at least one intraluminal image of the corresponding hollow organ section, for example, a bile duct, an esophagus or a section of the intestine, is captured using the imaging apparatus. The instrument 16 with the imaging apparatus, for example, a canulation instrument, a catheter or a guide wire with an OCT or IVUS apparatus, is located, for example, in the case of an ERCP, in the duodenum 13 in the vicinity of the opening 15 or already inside a section of the bile duct 9 or pancreatic duct 10. The intraluminal image or images depict a sphincter muscle, for example, the choledochal sphincter 11.1, the pancreatic sphincter 11.2, the papillary sphincter 11.3, or another sphincter muscle. Thus, more than one intraluminal image, for example, two or three images, may be captured, in particular from different projection directions, for example, also in a 360° view. Examples of intraluminal images that have been captured by an OCT apparatus from a hollow organ with a sphincter muscle are shown in FIG. 4, for example.

In act 21, sphincter muscle fibers and/or the structure and/or alignment of the sphincter muscle fibers of at least one sphincter muscle of the hollow organ section are detected, for example, segmented or otherwise determined or evaluated, on the basis of at least one image from the intraluminal image or images. Images such as the intraluminal OCT image shown in FIG. 4 may be used to detect and segment the sphincter muscle fibers or their alignment/orientation. Image processing algorithms may be used for segmentation, for example. Algorithms based on machine learning which have been trained beforehand with a multiplicity of images of sphincter muscle fibers may also be used. Sphincter muscle fibers may be ring-shaped. Additionally, or alternatively, a 3D reconstruction of the images may also be performed, e.g., if at least two intraluminal images have been captured.

In act 22, the current position and/or orientation of the instrument and/or the tip of the instrument, for example, the canulation instrument, the catheter or the guide wire, is also determined. This act may also be performed at the beginning of the method. Positioning may be determined using various known methods, for example, using sensors attached to the instrument or external sensors, or an external tracking system (for example, electromagnetic, RFID, etc.). When using a navigation robot for automatic movement of the instrument through the hollow organ (not part of the method), the sensor technology of the navigation robot may also be used for positioning.

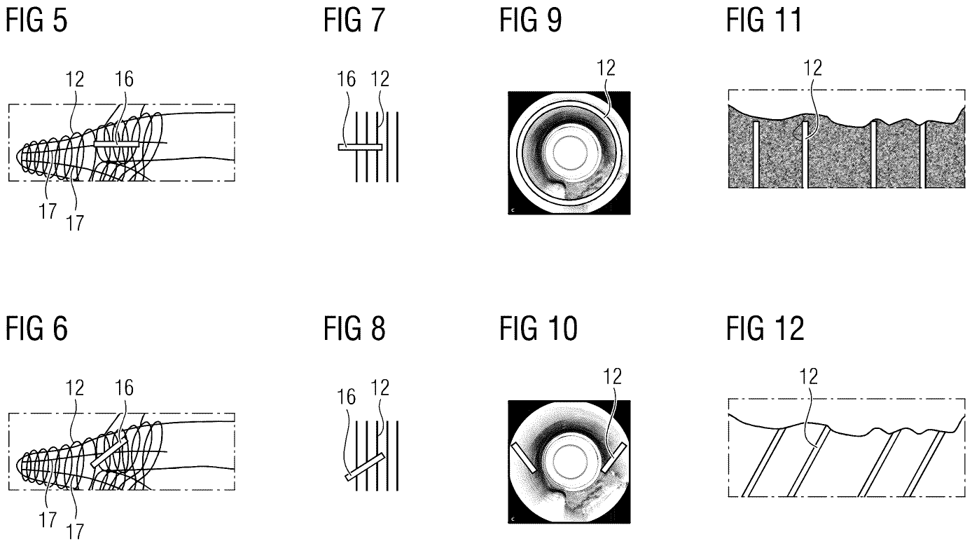

In act 23, a model and/or visualization of the sphincter muscle fibers of the hollow organ section relative to the current position and/or orientation of the instrument is determined and displayed. To determine the model, for example, a center line of the hollow organ section may be ascertained from the arrangement of the sphincter muscle fibers, and then visualization of the center line relative to the longitudinal axis of the instrument, for example, canulation instrument, (or the tip of the instrument) may be displayed. Sphincter muscle fibers may be ring-shaped and therefore have a center. Thus, for example, detected curved sections of muscle fibers (i.e., rings or parts of rings) may be used to deduce where the center point of the ring-shaped structure formed by them is located, and which orientation the ring-shaped structure has around this center point. A center line may be derived from the center points of at least two ring-shaped sphincter muscle fibers. Overall, a lot of information about the corresponding hollow organ section may be obtained from the ring-shaped sphincter muscle fibers. The model or visualization may be based on or use the intraluminal image or images captured, or may be formed from an abstract graphic, for example. Examples of this are shown in FIGS. 5 to 12.

FIGS. 5 to 8 show diagrammatic views of a series of sphincter muscle fibers in side view 12, which may have been created from intraluminal images and possibly other medical images, for example, pre-operative X-ray images. In FIGS. 7 and 8, the sphincter muscle fibers are shown only as lines. The instrument 16 or its longitudinal axis is also shown in the graphics. In FIGS. 5 and 7, the longitudinal axis of the instrument 16 is oriented along the center line 17, while in FIGS. 6 and 8 it deviates significantly therefrom.

FIGS. 9 to 12 are derived directly from the intraluminal images. Here, the relative orientation of the instrument may be seen from the fact that the image was obtained from the perspective of the instrument. FIGS. 9 and 10 depict an OCT image with the sphincter muscles shown accordingly. In FIG. 9, the perspective of the instrument is on the center line, which may be seen from the perfectly ring-shaped sphincter muscle fibers, while in FIG. 10 the sphincter muscle fibers are distorted, indicating that the instrument is deviating from the center line. FIGS. 11 and 12 show an image as it was captured, for example, during a pull-back movement or lateral movement. In FIG. 11, the image was captured along the center line, while in FIG. 12, it was captured deviating therefrom. The angle by which the sphincter muscle fibers in FIG. 12 deviate from the vertical or from the angle in FIG. 11 corresponds, for example, to the angle of the instrument (its longitudinal axis) to the wall of the sphincter muscle.

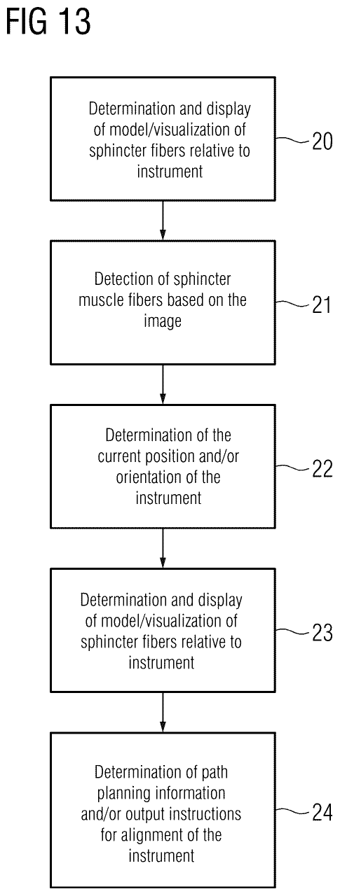

FIG. 13 shows a further sequence of acts of an image support method, the first four acts corresponding to those in FIG. 3. In act 24, path planning information is then determined for forward movement of the instrument e.g., along a predetermined center line of the hollow organ section, in particular with regard to the position and/or orientation that the instrument assumes for further path planning in order to continue moving according to the original plans. Alternatively, instructions for manual or semi-automatic alignment of the instrument may also be determined and issued, for example, instructions concerning the direction in and angle at which the instrument is rotated. Correction information for correcting the current path of the instrument may also be determined and output. In manual navigation, these may be instructions for the operator, which are output acoustically or as a display. In semi-automatic or automatic navigation, for example using navigation robots, control commands or signals may also be generated and transmitted.

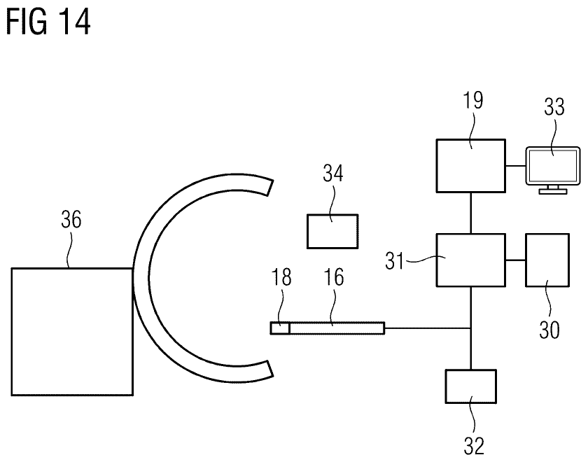

FIG. 14 shows a complete medical system 35 for performing the method. The complete medical system 35 has an instrument 16 for navigating in a hollow organ of a patient, including an intraluminal imaging apparatus 18, for example, an OCT apparatus or an IVUS apparatus, for capturing images inside a hollow organ. An imaging direction may, for example, be laterally oriented, i.e., in the direction of a wall of the hollow organ section, or along the center line of the hollow organ section; a plurality of imaging directions may also be used, and optionally, the instrument may be rotated for 360° lateral imaging (see FIG. 4).

Such imaging apparatuses 16 are generally known. The instrument 16 is, for example, a canulation instrument or a catheter, a guide wire may also be attached to the instrument, for example, to simplify navigation. The instrument may be controlled and moved, for example, manually, semi-automatically or automatically by a navigation robot 32 located outside the body. Such navigation robots 32 are known, for example, from the document “Evolution and current state of robotic catheters for endovascular surgery: A comprehensive review,” by N. V. Belikov et al., Engineering Science and Technology, an International Journal, Volume 57, September 2024, 101789. In addition, the complete system 35 has an image processing unit 19 with image processing software which is designed to detect sphincter muscle fibers and/or the alignment of sphincter muscle fibers of at least one sphincter muscle of the hollow organ section on the basis of at least one image, for example by segmentation or another image processing algorithm. Previously trained machine learning algorithms may also be used for this purpose.

In addition, the complete system 25 has a positioning unit 34 for determining the current position and/or orientation of the instrument and/or at least the tip of the instrument. Such a positioning unit may, for example, include one or more sensors which are arranged on the instrument and/or are arranged outside the body. For example, an electromagnetic tracking system based on magnetic location or transponder localization may be used, as known from the prior art (for example, article “Kabelloses elektromagnetisches Tracking in der Medizin,” A. M. Franz et al., Bildverarbeitung für die Medizin 2014, Informatik aktuell, 2014) [“Wireless electromagnetic tracking in medicine”, A. M Franz et al,. Imaging processing for medicine 2014, Informatik aktuell, 2014].

Other options include positioning by RFID, optical, ultrasound, or radio technology. Furthermore, the complete system has a calculation unit 30. This is designed, for example, to determine a model and/or visualization of the sphincter muscle fibers of the hollow organ section in particular relative to the current position and/or orientation of the instrument. For example, it may determine the corresponding rings from the segmented parts of sphincter muscle fibers or determine centers from the ring-shaped fibers and a center line of the hollow organ section from a plurality of center points. The determination relative to the current position and/or orientation of the instrument may also include a specific optical perspective without displaying the instrument, for example, as shown in FIGS. 9 to 12.

In addition, the complete system has a display unit for displaying the model or visualization, for example a monitor 33 or a touchpad with a display.

During the execution of the method, the canulation instrument is located in or immediately in front of a hollow organ section which is surrounded by ring-shaped sphincter muscle fibers.

A canulation instrument may optionally have a cutting apparatus with a cutting function (for example, needle knife, sphincterotome) which may be used to invasively dilate the bile duct section.

In addition, the complete system may include an X-ray device 36 for intraprocedural imaging (for example, a C-arm X-ray device, an angiography X-ray device, or a mobile X-ray device) which may be used to take X-ray images of the hollow organ section. These may be superimposed, for example, on the visualizations or displayed alongside them.

Optionally, pre-interventional 3D-data sets (e.g., MR/MRCP/CT), which depict the hollow organ sections, for example, bile ducts, may also be used for superimposition or navigation planning. Optionally, the data set also displays the ring-shaped sphincter muscle structure around the corresponding hollow organ sections. A 3D reconstruction of the X-ray images or other images may also be performed, and/or the intraluminal images may be registered with the X-ray images. Segmentations may also be transferred, for example, from the X-ray image coordinate system or a pre-interventional data set to the intraluminal images or vice versa. In particular, the position of the instrument, e.g., the tip of the imaging canulation instrument, may also be determined via X-ray imaging.

From the segmentation of the intraluminal images or additional image information, e.g., from pre-operative X-ray images or other X-ray images, a model of the current position and/or orientation of the muscle fibers relative to the current position of the instrument, e.g., the tip of the canulation instrument, is created, and the direction and/or center line of the current section of the hollow organ section is derived therefrom. Thus, for example, detected curved sections of muscle fibers may be used to deduce where the center of the ring-shaped structure formed by them is located and which orientation the ring-shaped structure has around this center. In radial OCT images, the “degeneration” of expected ring-shaped muscles may be detected (see FIGS. 9 and 10), and in pull-back or lateral imaging, the angle of the detected fibers corresponding to the angle of the devices to the hollow organ wall may be determined (see FIGS. 11 and 12).

The position of the center line of the hollow organ section may be superimposed on an X-ray image, for example, relative to the visible X-ray shadow of the tip of the canulation instrument. Instructions for optimized alignment of the instrument, for example, the tip of the instrument, may be issued in order to be able to follow the center line during subsequent navigation. Instructions may be issued for the subsequent triggering of a cutting function of a canulation instrument. If the instrument/tip of the instrument has been previously registered in a pre-interventional data set, the section of the hollow organ in which the tip of the instrument is currently located may be displayed.

The disclosure enables gentler canulation of hollow organs with sphincter muscles, for example, bile ducts, as it allows any deviation from the optimal alignment of the tip of the instrument with the bile duct to be detected and subsequently corrected.

The disclosure may be summarized as follows. For particularly gentle monitoring of navigation in hollow organs with sphincter muscles which are difficult to access, such as for example, a bile duct, a method for image support for navigation of an instrument including an imaging apparatus in a hollow organ section surrounded by at least one ring-shaped muscle or tissue structure, in particular a sphincter muscle, is provided. The method includes: capturing or providing at least one intraluminal image of the hollow organ section which was captured by the imaging apparatus; detecting the muscle or tissue fibers surrounding the hollow organ section, in particular sphincter muscle fibers, and/or alignment of the muscle or tissue fibers, in particular the sphincter muscle fibers, of at least one muscle or tissue structure, in particular the sphincter muscle, of the hollow organ section on the basis of at least one image; ascertaining the current position and/or orientation of the instrument and/or the tip of the instrument; and determining and providing a model and/or visualization of the muscle or tissue fibers, in particular sphincter muscle fibers, of the hollow organ section relative to the current position and/or orientation of the instrument.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A medical system configured to provide image support for navigation of an instrument in a hollow organ section surrounded by at least one ring-shaped muscle or tissue structure, the medical system comprising:
a canulation instrument configured to navigate in a hollow organ of a patient, the canulation instrument comprising an intraluminal imaging device;
an image processing unit configured to detect ring-shaped or directional muscle or tissue fibers surrounding the hollow organ section and/or an alignment of the muscle or the tissue fibers of the at least one ring-shaped muscle or the tissue structure of the hollow organ section based on at least one image;
a positioning unit configured to determine a current position and/or an orientation of the canulation instrument and/or a tip of the canulation instrument;
a calculation unit configured to determine a model and/or a visualization of the at least one ring-shaped muscle or the tissue structure of the hollow organ section relative to the current position and/or orientation of the canulation instrument; and
a provision unit configured to provide the model or the visualization.

2. The medical system of claim 1, wherein the at least one ring-shaped muscle or the tissue structure is a sphincter muscle, and
wherein the muscle or the tissue fibers are sphincter muscle fibers.

3. The medical system of claim 1, further comprising:
a display unit configured to display the model or the visualization.

4. The medical system of claim 1, further comprising:
a medical intervention robot configured to automatically control the canulation instrument.

5. The medical system of claim 1, further comprising:
an X-ray device configured to take X-ray images.

6. The medical system of claim 1, further comprising:
an algorithm or image processing software configured to determine a center line of the hollow organ section.

7. The medical system of claim 1, further comprising:
path planning software configured to determine path planning information for forward movement of the canulation instrument along a center line of the hollow organ section.

8. The medical system of claim 1, wherein the provision unit is configured to output instructions for alignment of the canulation instrument.

9. The medical system of claim 1, wherein the provision unit is configured to output correction information for correcting a current path of the instrument.

10. The medical system of claim 1, wherein the intraluminal imaging device is formed by an IVUS or OCT apparatus.

11. The medical system of claim 1, wherein the hollow organ section is formed by a bile duct, an esophagus, an arterial or venous blood vessel, or a section of intestine.

12. A method for image support for navigation of an instrument in a hollow organ section surrounded by at least one ring-shaped muscle or tissue structure, the method comprising:
capturing or providing at least one intraluminal image of the hollow organ section, which was captured by an imaging apparatus of the instrument;
detecting muscle or tissue fibers surrounding a hollow organ section and/or alignment of the muscle or the tissue fibers of the at least one ring-shaped muscle or the tissue structure of the hollow organ section based on the at least one intraluminal image;
ascertaining a current position and/or an orientation of the instrument and/or a tip of the instrument; and
determining and providing a model and/or a visualization of the muscle or the tissue fibers of the hollow organ section relative to the current position and/or the orientation of the instrument.

13. The method of claim 12, wherein the at least one ring-shaped muscle or the tissue structure is a sphincter muscle, and
wherein the muscle or the tissue fibers are sphincter muscle fibers.

14. The method of claim 12, wherein the determining and the providing comprises determining and providing of a center line of the hollow organ section.

15. The method of claim 12, further comprising:
determining path planning information for forward movement of the instrument along a center line of the hollow organ section for a position and/or orientation to be assumed by the instrument.

16. The method of claim 12, further comprising:
determining and outputting correction information for correcting a current path of the instrument.

17. The method of claim 12, further comprising:
triggering control signals for automatic alignment, movement, or action of the instrument by a medical intervention robot in an orientation to be assumed by the instrument.

18. The method of claim 12, further comprising:
registering the imaging apparatus with an X-ray device prior to the capturing or the providing of the at least one intraluminal image; and
displaying the at least one intraluminal image superimposed or together with an X-ray image captured by the X-ray device.

19. The method of claim 12, wherein at least two intraluminal images are captured and reconstructed to form a 3D volume image.

20. The method of claim 12, wherein further path planning information is determined for the instrument.
